(19) United States
(12) Patent Application Publication
(10) Pub. No.: US 2015/0352080 A1
(43) Pub. Date: Dec. 10, 2015

(54) BIOAVAILABLE COMPOSITIONS OF METAXALONE COMPRISING NONVOLATILE LIQUIDS AND PROCESSES FOR PRODUCING THE SAME

(71) Applicant: Spiridon Spireas, New Hope, PA (US)

(72) Inventor: Spiridon Spireas, New Hope, PA (US)

(21) Appl. No.: 14/832,580

(22) Filed: Aug. 21, 2015

Related U.S. Application Data
(63) Continuation of application No. 13/665,695, filed on Oct. 31, 2012, now abandoned.

(51) Int. Cl. A61K 31/421; A61K 9/20

(52) U.S. Cl. CPC A61K 31/421; A61K 9/2031; A61K 9/2095; A61K 9/2054; A61K 9/2059

(57) ABSTRACT

Pharmaceutical compositions comprising metaxalone which demonstrate improved dissolution and bioavailability characteristics compared to the commercially available product, and methods of producing them are provided. In a preferred embodiment, a dosage form comprising metaxalone and at least one inactive powder excipient is bioequivalent to its commercially available counterpart (Skelaxin® 400-mg tablets) after oral administration to fasting or non-fasting human subjects, while at the same time displaying faster drug dissolution rates than the Skelaxin® tablets as demonstrated from three different dissolution tests. In another preferred embodiment, a dosage form comprising metaxalone, at least one inactive powder excipient and a nonvolatile liquid is significantly more bioavailable than the commercially available Skelaxin® 400-mg tablets after oral administration to fasting human subjects.

### CROSS-REFERENCE TO RELATED APPLICATIONS

This application claims the benefits of U.S. Provisional Application No. 60/551,257, filed on Mar. 8, 2004, which is incorporated herein by reference in its entirety.

### FIELD OF THE INVENTION

The present invention relates to bioavailable pharmaceutical formulations of essentially water-insoluble drugs and processes for producing the same. In particular, the present invention relates to bioavailable, manufacturable, and stable pharmaceutical solid dosage forms comprising an essentially water-insoluble drug exemplified by metaxalone, a muscle-relaxant, and processes for producing the same.

### BACKGROUND OF THE INVENTION

Bioavailability of certain drugs, e.g., essentially water-insoluble drugs, can be limited when administered orally. Typically, manufacturers recommend that essentially water-insoluble drugs be taken along with food to enhance the bioavailability of the drugs.

For example, metaxalone or 5-[(3,5-dimethylphenoxy)methyl]-2 oxazolidinone is an essentially water-insoluble drug substance commercially available in the United States of America under the brand name Skelaxin® (trademark owned by Elan Pharmaceuticals, Inc. and distributed by King Pharmaceuticals), which is indicated as adjunct to rest, physical therapy and other measures for the relief of discomforts associated with acute, painful musculoskeletal conditions. Skelaxin® tablets containing 400 mg and 800 mg of metaxalone are presently available in the market.

Physical and chemical characteristics of metaxalone along with pharmacological, therapeutic and pharmacokinetic properties of the drug are reviewed in U.S. Pat. Nos. 6,407,128 and 6,683,102, which are incorporated herein by reference. As discussed in those patents and relevant literature cited therein, metaxalone is a hydrophobic, essentially water-insoluble powder, which demonstrates limited absorption from the gastrointestinal tract (GIT) when administered orally in the form of Skelaxin® tablets containing 400 mg metaxalone and other inert compression tabletting excipients. The two patents present a method to improve the oral bioavailability of metaxalone from the Skelaxin® tablet formulation by administering Skelaxin® tablets with food.

Apparently, the poor bioavailability of metaxalone is due to its low aqueous solubility resulting into a slow rate of drug dissolution in the aqueous contents of the GIT. According to the two patents listed above, the aqueous solubility of metaxalone and, hence, its dissolution rate and oral bioavailability are not enhanced by the formulation used to produce the Skelaxin® tablets. Consequently, the oral bioavailability of metaxalone from Skelaxin® tablets is improved only when it is administered with food wherein the lipids and other fats and substances contained in the food along with the excessive presence of bile salts and digestive enzymes in the GIT caused by the food, act as solubility enhancers of metaxalone thereby increasing the drug dissolution rate and oral bioavailability of Skelaxin® tablets.

Therefore, it would be beneficial to the extent and rate of drug absorption and, in general, to the oral bioavailability of essentially water-insoluble drugs, e.g., metaxalone, if specialty formulations were introduced that would enhance the solubility and dissolution rate of the drug without the need of co-administration of food. Hence, there is presently a need for manufacturable and stable solid dosage forms of metaxalone, which would possess improved oral bioavailability and/or drug dissolution rates as compared to the commercially available Skelaxin® tablets. In addition, it is also desirable for one to be able to manufacture bioavailable pharmaceutical tablet formulations of metaxalone which would demonstrate improved drug dissolution rates as compared to those of Skelaxin® tablets while at the same time, possessing similar bioavailability properties to those of the commercially available product, i.e., being bioequivalent to the Skelaxin® tablets after single dose oral administration to fasting and/or non-fasting human subjects.

International patent application with publication number WO-2004/019937 and a publication date of Mar. 11, 2004, deals with pharmaceutical compositions of metaxalone containing the drug in a micronized form wherein the metaxalone particles are reduced to levels below 10 micrometers (μm) of particle diameter. Specifically, 99% by volume of the particles possess particle diameters which are below a value of 10 μM. The authors of WO-2004/019937 suggest that their approach yields metaxalone 400-mg tablets with enhanced oral bioavailability as compared to commercially available Skelaxin® 400-mg tablets after single oral administration to nine (9) healthy male volunteers fasted overnight. However, the purported increase in oral bioavailability presented in that patent publication is marginal in terms of extent of absorption as defined by the Ln-transformed Test/Reference Ratio Percent of AUC_{inf} (defined in a later section of this specification). As shown in Table 8 of WO-2004/019937, said Test/Reference AUC_{inf} Ratio Percent did not even exceed the value of 125% which is widely accepted by regulatory agencies including the U.S. FDA as the upper limit of the 90% confidence intervals indicating significant difference between the oral bioavailability of the Test and Reference products. Furthermore, based on such results, the authors of WO-2004/019937 claim that using their micronized metaxalone tablet formulation would not present a food effect on oral bioavailability. However, the marginal increase in the Test/Reference AUC_{inf} Ratio Percent observed in those studies implies exactly the opposite. In other words, it should be expected that, as reported in U.S. Pat. Nos. 6,407,128 and 6,683,102 wherein the Skelaxin® reference tablets were dosed under fasting and non-fasting conditions, the metaxalone tablets of WO-2004/019937 which demonstrated an extent of absorption quite similar to that of the Skelaxin® 400-mg tablets under fasting dosing conditions, should also show a significant food effect on oral bioavailability which should be quite similar to that of the commercially available Skelaxin® 400-mg tablets.

As such, there remains a long-standing need for improved bioavailable formulations of essentially water-insoluble drugs and methods for preparation of the same. An object of the present invention is to provide pharmaceutical compositions comprising an essentially water-insoluble drug, e.g., metaxalone, and at least one inactive ingredient, which demonstrate improved drug dissolution rates as compared to the commercially available product of the same essentially water-insoluble drug, e.g., Skelaxin®. Another object is to provide pharmaceutical compositions comprising an essentially water-insoluble drug, e.g., metaxalone, and at least one inactive ingredient, which demonstrate improved drug dissolution rates and truly enhanced bioavailability properties as compared to the commercially available product of the same essentially water-insoluble drug, e.g., Skelaxin®. Furthermore, another object of the present invention is to provide pharmaceutical compositions comprising an essentially water-insoluble drug and at least one inactive ingredient, which demonstrate maximally enhanced bioavailability properties under fasting and non-fasting dosing conditions as compared to conventionally made formulations or the commercially available product of the same essentially water-insoluble drug. Finally, another object of this invention is to provide pharmaceutical compositions comprising an essentially water-insoluble drug and at least one inactive ingredient, which do not only demonstrate maximally enhanced bioavailability properties as compared to conventionally made formulations or the commercially available product of the same essentially water-insoluble drug, but they also present none or no significant food effect on the drug's oral bioavailability, i.e., they demonstrate similar bioavailability properties when dosed to fasting and non-fasting human subjects in contrast to conventional or commercial formulations of the drug.

### SUMMARY OF THE INVENTION

Pharmaceutical compositions prepared according to the present invention comprise essentially water-insoluble drugs, e.g. metaxalone, treated with various combinations of sparingly water-soluble or essentially water-insoluble, natural or synthetic hydrocolloids and polymers with or without the use of several volatile liquids and/or nonvolatile liquids, while at the same time demonstrating certain desirable drug dissolution rates and oral bioavailability.

The practice of the present invention employs, unless otherwise indicated, conventional methods of chemistry and drug synthesis and formulation, all within the skill of the art. Such techniques are explained fully in the literature. See, e.g., Remington: The Science and Practice of Pharmacy by Alfonso R. Gennar, editor, (19th edition 2000), incorporated herein by reference.

### BRIEF DESCRIPTION OF THE DRAWINGS

FIG. 1 shows the summary of statistical analysis of dosing of metaxalone under non-fasting conditions in the forms of the Ex.#2 metaxalone 400-mg tablets (Lot # BB5800087) dosed as the Test product and commercial Skelaxin® 400-mg tablets (Lot # GS1109A) dosed as the Reference product.

FIG. 2 shows the summary of statistical analysis of dosing of metaxalone under fasting conditions in the forms of the Ex.#2 metaxalone 400-mg tablets (Lot # BB5800087) dosed as the Test product and Skelaxin® 400-mg tablets (Lot # GS1109A) dosed as the Reference product.

FIG. 3 shows the mean plasma concentration (ng/mL) from time 0 hours to time 36 hours after dosing of metaxalone under fasting conditions in the forms of the Ex.#2 metaxalone 400-mg tablets (Lot # BB5800087) dosed as the Test product and commercial Skelaxin® 400-mg tablets (Lot # GS1109A) dosed as the Reference product.

FIG. 4 shows the mean plasma concentration (ng/mL) from time 0 hours to time 36 hours, on a semi-logarithmic scale, after dosing of metaxalone under fasting conditions in the forms of the Ex.#2 metaxalone 400-mg tablets (Lot # BB5800087) dosed as the Test product and commercial Skelaxin® 400-mg tablets (Lot # GS1109A) dosed as the Reference product.

FIG. 5 shows the mean plasma concentration (ng/mL) from time 0 hours to time 36 hours after dosing of metaxalone under fasting conditions in the forms of the novel, maximally bioavailable Ex.#4 metaxalone 400-mg tablets (Lot # BB5800056) dosed as the Test product and commercial Skelaxin® 400-mg tablets (Lot # GS803A) dosed as the Reference product.

### DETAILED DESCRIPTION OF ILLUSTRATIVE EMBODIMENTS

Overall, the present invention relates to compositions of essentially water-insoluble drugs, e.g., metaxalone, which, in contrast to commercial formulations, e.g., the Skelaxin® tablets, demonstrate improved “drug dissolution rate” and/or an “oral bioavailability” equivalent to, or greater than, that of the commercially available tablet formulations. Skelaxin® (400 mg strength) was been approved by the FDA under NDA #13-217 prior to Jan. 1, 1982. In 2002, supplemental new drug applications NDA #13-217/S-044 (400 mg) and NDA#13-217/S-036 (800 mg), which relate to administering Skelaxin® with food, were approved.

The term “drug dissolution rate” is defined herein as the amount or percent by weight of an essentially water-insoluble drug, e.g., metaxalone, dissolved within a given time period, namely, within the first 30 minutes of dissolution, from a unit of a solid dosage form, for example, from one tablet containing 400 mg of metaxalone being subjected to a specific dissolution test, which is characterized by a certain volume and type of the employed dissolution liquid medium that was maintained at a certain temperature and agitated by a given speed and type of a certain steering device. In the present studies, three dissolution tests (i.e., Tests A, B and C) were used to assess and compare the dissolution properties of the products evaluated. Such dissolution tests are described in a later section of this specification.

The term “oral bioavailability” is defined herein as the measure of the rate and extent of drug absorption in healthy human volunteers as expressed by Cₘₐₓ, which is the average maximum metaxalone concentration in plasma obtained during each study from human subjects, and AUC_{inf}, which is the average area under the metaxalone plasma concentration over time curve, obtained after a single dose oral administration of a drug product, for example, 400-mg metaxalone tablets (Skelaxin® or experimental tablets) during two period crossover clinical studies under fasting or non-fasting conditions. For the work related to the present invention, several two-period crossover relative bioavailability studies using various numbers of subjects ranging from 43 to 8 healthy male volunteers were conducted to compare the oral bioavailability of Skelaxin® reference tablets to that of experimental metaxalone test tablets. The methodology and statistical analysis employed in each of these biostudies are described in detail in a later section of this specification. Overall, in each of the biostudies performed, a given Test product (i.e., experimental metaxalone 400-mg tablets) was compared to the Reference product (i.e., commercial Skelaxin® 400-mg tablets) by using ANOVA treatments to assess the geometric means and upper and lower 90% confidence interval (CI) limits of the individual Test/Reference ratio percents of AUC_{inf} and Cₘₐₓ based on non-transformed and ln-transformed drug plasma concentrations obtained at various time intervals after single oral administration of a given Test or Reference product to each subject.

It should be specified that, in the work related to this invention, a given experimental metaxalone tablet formulation tested, was considered to be “bioequivalent” to the reference Skelaxin® tablets of NDA #13-217 if both of the obtained ln-transformed mean Test/Reference AUC_{inf} and Cₘₐₓ ratio percents along with their corresponding lower and upper CI limits were within a lower limit of 80% and an upper limit of 125%. On the other hand, experimental metaxalone products presenting both non-transformed and ln-transformed mean Test/Reference Cₘₐₓ, AUC₀₋ₜ and AUC_{inf} ratio percents greater than an upper limit of 125% were considered to be “more bioavailable” than the reference Skelaxin® tablets of NDA #13-217. In addition, in the case that experimental metaxalone tablet formulations presented both non-transformed and ln-transformed mean Test/Reference Cₘₐₓ, AUC₀₋ₜ and AUC_{inf} ratio percents greater than 150%, such products were considered to be “significantly more bioavailable” than the reference Skelaxin® tablets of NDA #13-217. Such “significantly more bioavailable” experimental metaxalone tablet formulations are unique according to the present invention and they are also referred to herein as “maximally bioavailable” compositions. Finally, ln-transformed mean Test/Reference AUC_{inf} and/or Cₘₐₓ ratio percents smaller than the lower CI limit of 80% led to concluding that such tested metaxalone tablets were “less bioavailable” than the Skelaxin® tablets of NDA #13-217.

Based on the metaxalone tablet formulations developed in connection to this invention, it has been now discovered that pharmaceutical compositions containing metaxalone treated with various combinations of sparingly water-soluble or essentially water-insoluble, natural or synthetic hydrocolloids and polymers with or without the use of various volatile and/or nonvolatile liquids, can be prepared demonstrating significantly improved oral bioavailability and/or drug dissolution rates of metaxalone as compared to the commercial Skelaxin® tablets.

Hydrocolloids and polymers preferred to be employed in the metaxalone solid dosage forms according to the present invention include, but are not limited to, alginic acid and its salt derivatives such as sodium, ammonium and calcium alginates, etc. (collectively termed herein as “alginate excipients” or “alginate powder excipients”), cellulosic derivatives such as sodium carboxymethyl cellulose, hydroxypropylmethyl cellulose, hydroxypropyl cellulose, ethyl cellulose, etc., copolymers of acrylic acid such as methacrylic acid copolymer, etc., various types and grades of polyvinyl pyrrolidones, and other natural, semi-synthetic or synthetic hydrocolloids and/or other polymers which may improve the aqueous solubility of metaxalone and/or the drug's wetting properties.

Volatile liquids preferred to be used in preparing the metaxalone solid dosage forms according to the present invention include, but are not limited to, methanol, ethanol, acetone, water, or combinations thereof. On the other hand, nonvolatile liquids preferred to be used in preparing the metaxalone solid dosage forms according to the present invention include, but are not limited to, cosolvents such as liquid or semisolid polyethylene glycols, propylene glycol glycerin, pharmasolve, etc., liquid or semisolid surface active agents such as liquid or semisolid polysorbates, cremophors, spans, myglyols, pluronics, etc., and other oils and oily liquids such as fish oil, olive oil, castor oil, vitamin-E, lecithin, etc., or combinations thereof that may improve the aqueous solubility of metaxalone and/or the drug's wetting properties.

As used herein, the term “nonvolatile liquid” means any liquid which, at sea level and standard pressure conditions (i.e., atmospheric pressure equal to 1 atm) possesses a boiling point greater than the boiling point of distilled water, namely, a boiling point greater than 100° C. On the other hand, the term volatile liquid means any liquid which at sea level and at 1 atm pressure possesses a boiling point equal to or less than 100° C. Those of skill in the art will understand that whenever a volatile liquid is used to wet a powder admixture serving to promote a granulation process, it would be subsequently removed from the final granulation powder blend by means of drying. This, however, does not occur when nonvolatile liquids are incorporated into the formulation. In such case, the nonvolatile liquid does not evaporate during drying and it remains within the final dosage form for the life of the products. Pharmaceutical compositions containing nonvolatile liquids are termed Liquisolid Systems and are described in U.S. Pat. No. 5,800,834, U.S. Pat. No. 5,968,550, U.S. Pat. No. 6,096,337 and U.S. Pat. No. 6,423,339, which are incorporated herein by reference.

Other than the preferred ingredients discussed above, metaxalone compositions according to the present invention may also contain various inactive pharmaceutical excipients. Those of skill in the art will understand that the term “excipient” is used colloquially to include such pharmaceutical adjuvants as fillers, diluents, binders, disintegrants, glidants, lubricants, pigments, colorants, coating agents, lubricants and the like. “Fillers” and “diluents” are powders used to add volume to the drug/powder blends in order to improve the distribution, uniformity, stability and processing of the drug in the final product. “Binders” are agents that hold the components of the formulation together. “Disintegrants” are agents that enhance the conversion of a compact material into fine primary particles during the dissolution of the final product. “Glidants” are additives that reduce particle friction and induce good flowing properties of the final drug/powder blend. “Lubricants” are additives used to prevent the sticking of the product formulation to tooling during the tabletting process. It will be appreciated by persons of ordinary skill in the art that such inactive pharmaceutical materials should be consistent with the overall spirit of the invention. Thus, such materials may be employed which do not adversely effect the processing set forth herein and which do not interfere with the stability of the resulting products.

Therefore, the term “excipient,” “powder excipient,” or “inactive powder excipient” as used herein include, but is not limited to: “hydrocolloids and polymers” discussed previously such as the “alginate powder excipients” (i.e., alginic acid and sodium, ammonium and calcium alginates), cellulosic derivatives, copolymers of acrylic acid, polyvinyl pyrrolidones and other natural, semi-synthetic or synthetic hydrocolloids and polymers; “cellulosic powder excipients” such as microcrystalline cellulose, powder and amorphous cellulose, carboxymethyl and sodium carboxymethyl cellulose, methyl and ethyl cellulose, hydroxyethyl cellulose and hydroxypropyl cellulose, hydroxypropylmethyl cellulose and other cellulosic derivatives and modified celluloses; “starch powder excipients” such as corn starch, maze starch, potato starch, pregelatinized starch, sodium starch glycolate and other starch derivatives; “sugar-base powder excipients” such as lactose, sucrose, maltose, dextrose, fructose, cyclodextrins, etc.; and “other powder adjuvants” such as dicalcium and tricalcium phosphate, amorphous silicon dioxide, fumed silica, talc, croscaramelose sodium, povidone, crosslinked povidone, magnesium stearate, stearic acid, glyceryl monostearate, hydrogenated vegetable oil, waxes and other inert powders including stabilizing, coloring and coating agents, that are useful for tabletting or encapsulation.

By “pharmaceutically acceptable” or “pharmacologically acceptable” is meant a material which is not biologically or otherwise undesirable, i.e., the material can be administered to an individual without causing any undesirable biological effects or interacting in a deleterious manner with any of the components of the formulation in which it is contained.

In some preferred embodiments of this invention, the drug is dry-mixed with certain inactive and pharmaceutically acceptable powder excipients. The drug/powder blend is then wetted and granulated with certain pharmaceutically acceptable volatile liquids with or without the presence of certain pharmaceutically acceptable nonvolatile liquids. The wet granulation is then dried, milled and subsequently mixed with other powder excipients to yield the final drug/powder blend which is then compressed into tablets to produce the pharmaceutical composition or final solid dosage form.

As used herein, the term “drying” refers to the substantial removal of the volatile granulating liquid from the granulation. Drying may be accomplished in a number of manners well known to those of skill in the art including, but not limited to the use of ovens, fluid bed driers, and other similar equipment. In a preferred embodiment, the granulation is dried in an oven for about 8 hours at 50° C. to substantially remove the volatile liquid from the granulation. In another preferred embodiment, the granulation is dried in an oven for about 10 hours at 50° C. to substantially remove the volatile liquid from the granulation.

The processes of mixing, granulating, drying, milling, compressing, coating and making pharmaceutical solid formulations are well known to those of skill in the art. See, e.g., Theory & Practice of Industrial Pharmacy, 3rd Edition, Liberman, Lachman, and Kanig, eds. (Philadelphia, Pa.: Lea & Febiger), incorporated herein by reference.

In this work, several metaxalone tablet formulations were prepared and tested for their drug dissolution rates using three different dissolution tests discussed below. Furthermore, four of the experimental metaxalone products were tested by conducting relative bioavailability studies using commercially available Skelaxin 400-mg tablets as the reference product. The bioavailability properties of such Test metaxalone products where compared to the Reference Skelaxin® tablets using various numbers of healthy male volunteers in single dose two-period crossover studies under fasting and non-fasting dosing conditions as discussed below.

### Example Formulations

The experimental metaxalone tablet formulations prepared and tested are listed in Table 1. In general, metaxalone formulations were made by dry-mixing metaxalone powder with inactive powdered ingredients. Then, the resulting powder blends were wet-granulated using various volatile liquids with or without the presence of nonvolatile liquids. After drying and milling the granulation, other powder excipients were blended with the granulated powder to produce the final metaxalone product blend which was compressed into tablets each containing 400-mg of metaxalone. It should be noted that when nonvolatile liquids were incorporated in the final dosage forms as in the experimental metaxalone formulations Ex.#4 and Ex.#5 shown in Table 1 below, techniques and compositions related to the principles of liquisolid technology and systems were employed. Liquisolid systems and methods of producing same are discussed in U.S. Pat. No. 5,800,834, U.S. Pat. No. 5,968,550, U.S. Pat. No. 6,096,337 and U.S. Pat. No. 6,423,339, which are incorporated herein by reference. The ingredients of the experimental metaxalone tablet formulations prepared herein are listed in Table 1.

TABLE 1
Ingredients and Other Characteristics of Prepared
Experimental Metaxalone Tablet Formulations (Ex. # 1-5).
EXPERIMENTAL METAXALONE TABLET FORMULATIONS

| Formulation Ingredients | Ex. # 1 | Ex. # 2 | Ex. # 3 | Ex. # 4 | Ex. # 5 |
|---|---|---|---|---|---|
| (mg/tablet) | (BB5800040) | (BB5800087) | (BB5800047) | (BB5800056) | (NB1190:48) |
| Metaxalone (non-micronized) | 400 | 400 | 400 | 400 | 400 |
| Sodium Carboxy- | 10 | — | 15 | 39 | 15 |
| Methyl Cellulose | | | | | |
| Methacrylic Acid | — | — | — | 25 | — |
| Copolymer | | | | | |
| Ammonium Alginate | — | 21.5 | — | — | — |
| Sodium Alginate | — | 3 | — | — | — |
| Polyethylene Glycol 400 | — | — | — | 2 | — |
| Polysorbate 80 | — | — | — | — | 5 |
| Microcrystalline Cellulose | 65 | 30 | 66 | 33.5 | 66 |
| Pregelatinized Starch | 13.5 | 35 | 5.5 | — | 5.5 |
| Sodium Starch Glycolate | 7 | — | 4 | 4 | 4 |
| Magnesium Stearate | 4 | — | 4 | 4 | 4 |
| Stearic Acid | — | 4 | — | — | — |
| Red Iron Oxide | 0.5 | 0.5 | 0.5 | 0.5 | 0.5 |
| Denatured Alcohol | — | (14) | (35) | (79) | (35) |
| (granulation liquid - not | | | | | |
| present in the final product) | | | | | |
| Purified Water | (130) | (130) | (155) | (15) | (155) |
| (granulation liquid - not | | | | | |
| present in the final product) | | | | | |
| Tablet Weight | 500 | 494 | 495 | 508 | 500 |

Dissolution Studies:

Three different dissolution tests (Test-A, -B and -C) based on a modified USP dissolution apparatus II (paddle method) were used to assess the drug dissolution profiles of each of the experimental metaxalone 400-mg tablet formulations (i.e., Ex.#1-5) prepared herein and commercially available lots of Skelaxin® 400-mg tablets. In dissolution Test-A, each 400-mg metaxalone tablet (Skelaxin® or experimental tablet) was placed into a glass peak vessel filled with 1000 mL of purified water which was maintained at room temperature (i.e., 25° C.) and stirred at a paddle speed of 100 rpm. In dissolution Test-B, each 400-mg metaxalone tablet was placed into a standard glass vessel filled with 1000 mL of purified water which was maintained at 35° C. and stirred at a paddle speed of 100 rpm. Finally, in dissolution Test-C, each 400-mg metaxalone tablet was placed into a glass peak vessel filled with 500 mL of a 0.1% w/v sodium lauryl sulfate (SLS) solution in water, which was maintained at 37° C. and stirred at a paddle speed of 50 rpm. The metaxalone 30-minute drug dissolution rates (i.e., % drug dissolved within the first 30 minutes of dissolution) obtained from the five experimental metaxalone tablet formulations (Ex.#1-5) and commercial lots of Skelaxin® 400-mg tablets using dissolution Test-A, Test-B and Test-C, are compared in Table 2.

TABLE 2
Metaxalone Drug Dissolution Rates (% drug dissolved in the first 30
minutes) obtained from experimental metaxalone tablet formulations
(Ex. #1-5) and commercial Skelaxin ® Tablets using 3 different
dissolution tests (i.e., Test-A, Test-B and Test-C).
METAXALONE TABLET FORMULATIONS

| Drug Dissolution Rate | Ex. # 1 | Ex. # 2 | Ex. # 3 | Ex. # 4 | Ex. # 5 | SKELAXIN ® |
|---|---|---|---|---|---|---|
| (% Drug Dissolved in the | (BB5800040) | (BB5800087) | (BB5800047) | (BB5800056) | (NB1190:48) | (Com. Lots) |
| first 30 minutes of dissolution) | 400-mg Tablets | 400-mg Tablets | 400-mg Tablets | 400-mg Tablets | 400-mg Tablets | 400-mg Tablets |
| Dissolution Test-A | 9% | 29% | 34% | 39% | 46% | 13% |
| Peak Vessels - 1000 mL Water | | | | | | |
| (25° C.) - 100 rpm Paddles | | | | | | |
| Dissolution Test-B | 22% | 40% | 48% | 52% | 57% | 28% |
| Std Vessels - 1000 mL Water | | | | | | |
| (35° C.) - 100 rpm Paddles | | | | | | |
| Dissolution Test-C | 13% | 30% | 37% | 42% | 48% | 27% |
| Peak Vessels - 500 mL 0.1% | | | | | | |
| SLS (37° C.) - 50 rpm Paddles | | | | | | |

Bioavailability Studies:

Five relative bioavailability (bioequivalence) studies were conducted under fasting (four studies) or non-fasting (one study) dosing conditions using each of the four experimental metaxalone tablets formulations, namely, Ex.#1-4, manufactured by Mutual Pharmaceutical Company, Inc. as the Test product and commercially available Skelaxin® 400-mg tablets manufactured by Mallinckrodt, Inc. for Carnrick Laboratories, Inc. (Division of Élan Pharmaceuticals) as the Reference product. Specifically, Ex.#1 (Lot # BB5800040) metaxalone 400-mg tablets were tested against Lot # GS639A of Skelaxin® 400-mg tablets under fasting dosing conditions on 35 healthy male volunteers. Ex.#2 (Lot # BB5800087) metaxalone 400-mg tablets were tested against Lot # GS 1109A of Skelaxin® 400-mg tablets under fasting and non-fasting conditions on 43 and 24 healthy male volunteers, respectively. Ex.#3 (Lot # BB5800047) metaxalone 400-mg tablets were tested against Lot # GS639A of Skelaxin® 400-mg tablets under fasting conditions on 24 healthy male volunteers. Finally, Ex.#4 (Lot # BB5800056) metaxalone 400-mg tablets were tested against Lot # GS803A of Skelaxin® 400-mg tablets under fasting dosing conditions on 8 healthy male volunteers. In each study, a randomized, two-way crossover design was used to compare the relative bioavailability (rate and extent of drug absorption) of the Test (A) and Reference (B) products. The human subjects participating in each study were initially randomly assigned a sequence AB or BA. A single oral dose of the Test (A) and Reference (B) product was administered to the volunteers on two separate occasions under fasting or, in one study involving Ex.#2, non-fasting conditions, with at least a 7-day washout period between doses. Food and fluid intake were controlled during each confinement period.

Depending on the biostudy, about 18 to 23 blood samples per subject were collected each period at prescheduled time intervals spanning from time 0 hours (obtained within one hour prior to dose administration) to 36 or 48 hours for drug content analysis. Slight deviations from some scheduled times of sample collection were acceptable and, in such case, the actual time intervals were used in the statistical analysis. After analytical determination of the metaxalone plasma concentrations in each sample by the Analytical Laboratory of PRACS Institute, Ltd., the results were evaluated by the Statistical Division of PRACS Institute, Ltd. in order to calculate the pharmacokinetic parameters using WinNonlin™, Version 3.1, software designed specifically for analyzing pharmacokinetic data. WinNonlin™ Model 200 for extra-vascular input was utilized. All other computations were completed using SAS®, Version 8.2 software for Windows.

The following pharmacokinetic parameters were computed from the plasma concentration data using the actual sample collection times:

| | |
|---|---|
| AUC₀₋ₜ | Area under the plasma concentration-time curve (ng-hr/mL) |
| | from time zero to the time of the last quantifiable concentration |
| | (t), calculated using the linear trapezoidal rule: |
| | Σᵢ (tᵢ − t_{i - 1})(Cᵢ + C_{i - 1})/2, i = 1 to t, |
| | where Cᵢ is the plasma concentration at time tᵢ. |
| AUC_{inf} | Area under the plasma concentration curve from time zero |
| | extrapolated to infinity (ng-hr/mL), calculated by AUC₀₋₁ + |
| | (Cₗₐₛₜ/kₑₗᵢₘ), where Cₗₐₛₜ is the last quantifiable concentration and |
| | kₑₗᵢₘ is the terminal elimination rate constant. |
| Cₘₐₓ | Maximum or peak concentration, obtained by inspection |
| | (ng/mL). |
| Tₘₐₓ | Time of maximum or peak concentration, obtained by |
| | inspection (hr). |
| kₑₗᵢₘ | Terminal elimination rate constant (1/hr). This value was |
| | estimated by linear regression on the terminal phase of the |
| | semi-logarithmic concentration versus time curve. |
| t_{1/2} | Half life of the product (hr), calculated by ln(2)/kₑₗᵢₘ. |

Natural logarithmic (ln) transformations were computed for Cₘₐₓ, AUC₀₋ₜ and AUC_{inf}. In this way, the geometric means of the ln-transformed Least Squares Means were calculated.

In each study, an analysis of variance (ANOVA) was performed on each of the pharmacokinetic parameters using SAS® software. The ANOVA model containing factors for sequence of products, subjects within sequence, periods and products was utilized in comparing the effects between the Test and Reference products. Differences were declared statistically significant at the 5% level. A 90% confidence interval about the ratio of the mean test value to mean reference value was calculated for all of the pharmacokinetic parameters. The power of the ANOVA to detect a difference equal to 20% of the reference mean was also calculated with the SAS® software. The calculations for the power and confidence interval used the least squares means (LSMEANS) and the standard error of the estimate, both generated by the SAS® software. The ratio of the geometric means for the ln-transformed data and the corresponding 90% confidence intervals were calculated for AUC₀₋ₜ, AUC_{inf}, and Cₘₐₓ, as well. It should be noted that the lower limit of quantitation for metaxalone was 10.00 ng/mL. For statistical analysis, subject sample values below the lower limit of quantitation (BLQ) were reported as zero.

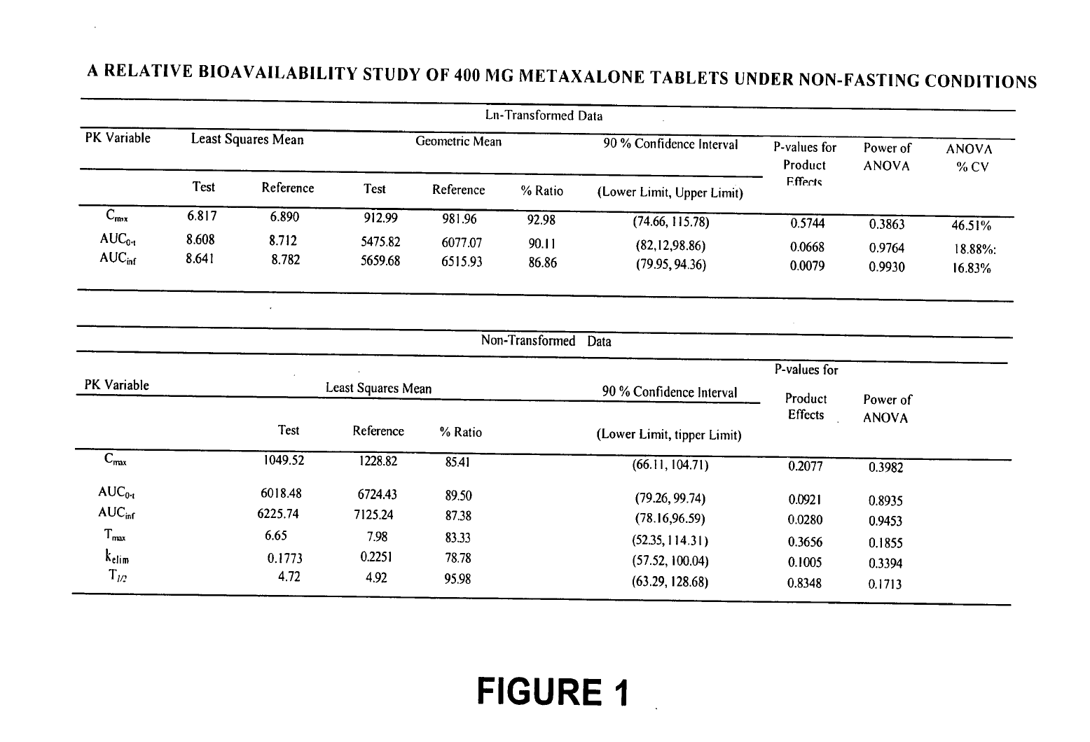

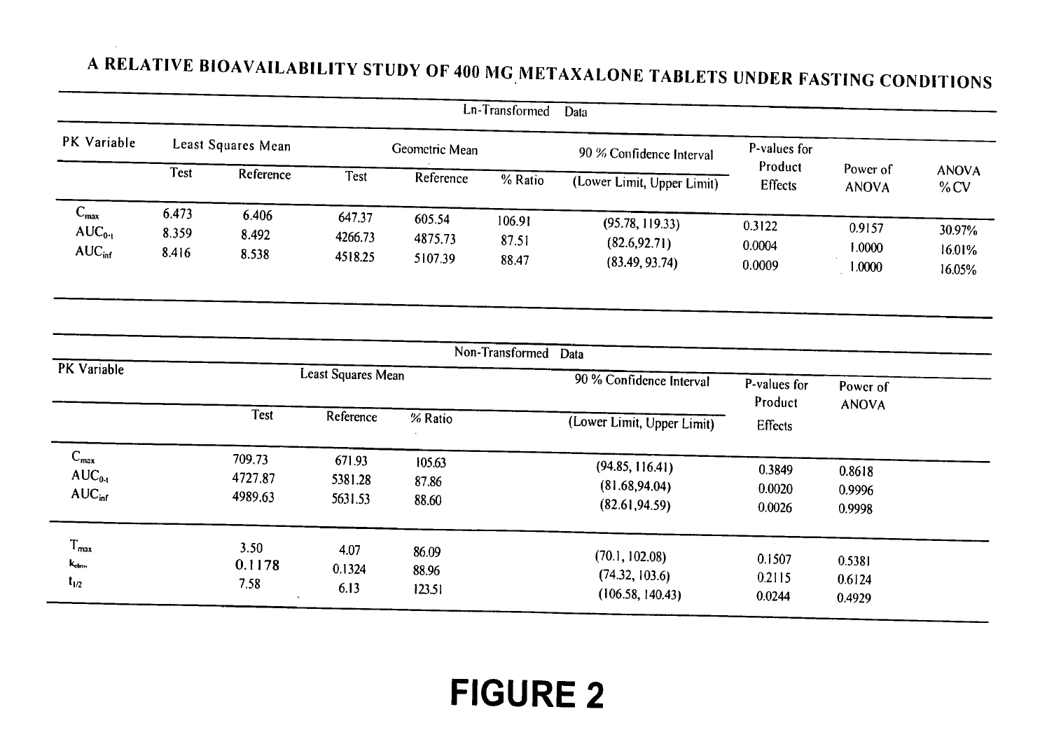

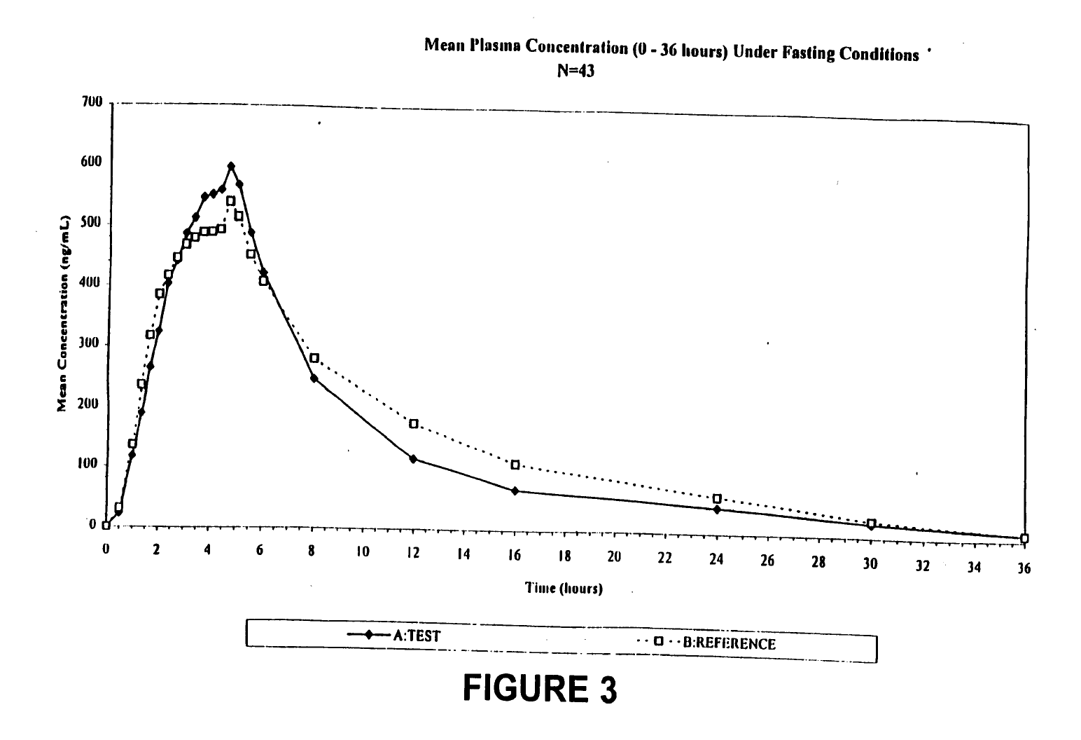

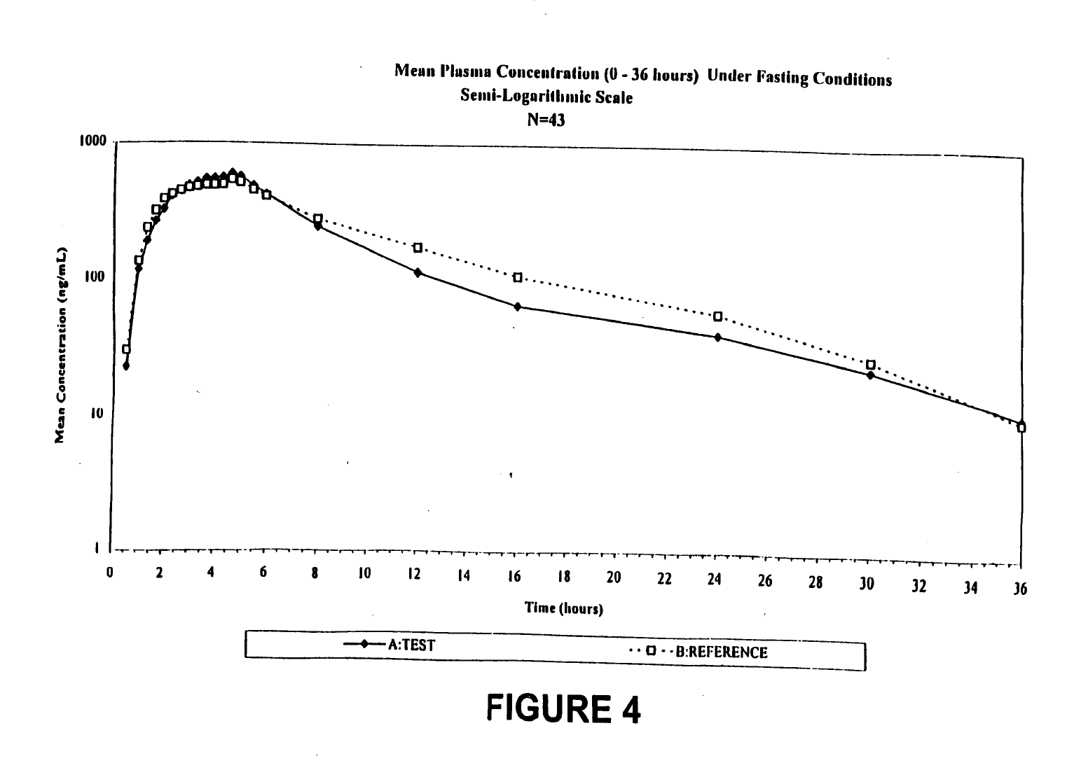

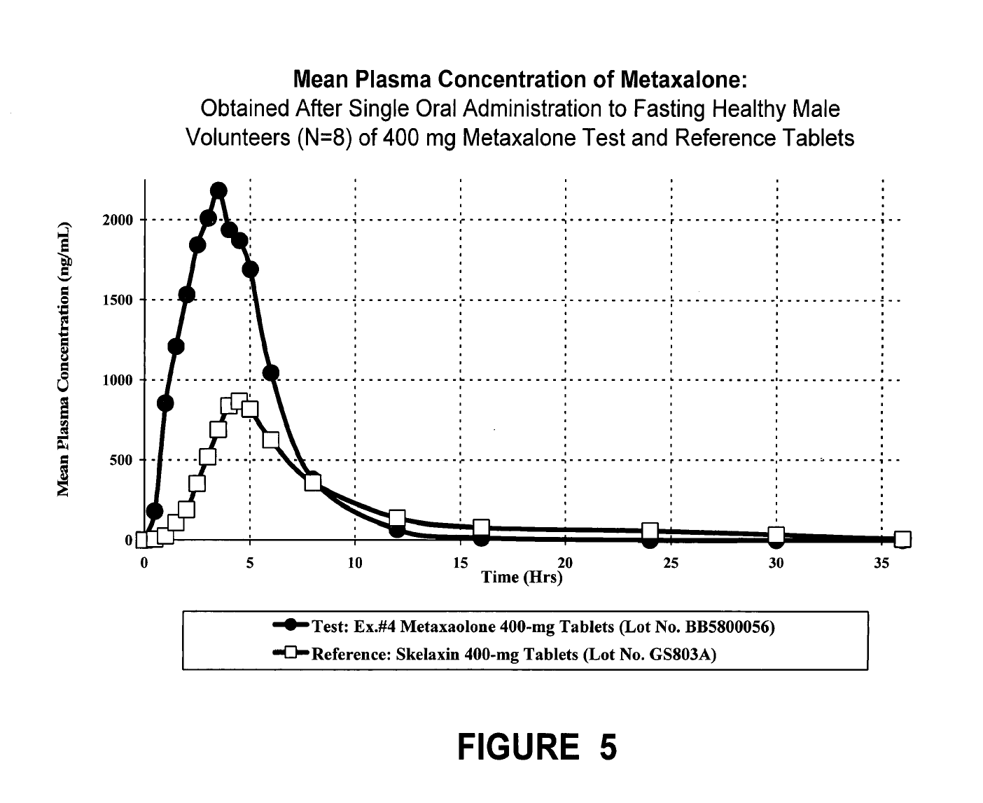

Results of non-transformed and ln-transformed computations of the critical pharmacokinetic parameters and their corresponding Test/Reference ratio percents obtained from each study are given in Tables 3 and 4, respectively, where it can be seen that the Test metaxalone tablets are “less-bioavailable” (i.e., Ex.#1), “bioequivalent” (i.e., Ex.#2, at fasting and non-fasting dosing conditions), “more-bioavailable” (i.e., Ex.#3), or “significantly-more-bioavailable” (i.e., Ex.#4) than the Reference Skelaxin® 400-mg tablets. The statistical analysis summary Tables related to the fasting and non-fasting bioequivalence studies conducted using Ex.#2 metaxalone 400-mg tablets (Lot # BB5800087) as the Test product and Skelaxin® 400-mg tablets (Lot # GS1109A) as the Reference product are shown in FIGS. 1 and 2. Furthermore, the absorption profiles (i.e., curves of the mean plasma concentrations of metaxalone obtained from all subjects in each time interval against time) after single oral administration of experimental metaxalone tablets Ex.#2 (both normal and semilogarithmic plots) or Ex.#4 (only normal plot) dosed as the Test products against the Reference Skelaxin® tablets are compared in FIGS. 3, 4 and 5.

TABLE 3
Comparison of oral bioavailability of metaxalone as defined by the values of the
non-transformed Least Squares Means of critical pharmacokinetic parameters and
the non-transformed Mean Test/Reference ratio percents of Cₘₐₓ, AUC_{inf}, AUC₀₋ₜ,
Tₘₐₓ, kₑₗᵢₘ and t_{1/2} obtained from clinical biostudies on healthy human volunteers
in two period crossover studies involving a single dose oral administration under
fasting conditions of each of the four experimental metaxalone tablet formulations
(Ex. #1-4) used as the Test product versus Skelaxin ® 400-mg tablets used as the
Reference product. The projected values expected to be obtained from a similar
bioequivalence study comparing the fifth experimental tablet formulation (Ex. #5)
against the Reference Skelaxin ® 400-mg tablets are also tabulated.
METAXALONE TABLET FORMULATIONS Tested Against SKELAXIN 400-mg Tablets

| Non-transformed Data: | | | | | |
|---|---|---|---|---|---|
| Non-transformed Least | | | | Ex. # 4 | Ex. # 5 |
| Squares Means of Critical | | | | (BB5800056) | (NB1190:48) |
| PK Parameters and | | | | Significantly More | Projected to be |
| Test/Reference Ratios | | | | Bioavailable Than | Significantly More |
| Means of Non-Transformed Least | Ex. # 1 | Ex. # 2 | Ex. # 3 | Skelaxin Reference | Bioavailable Than |
| Squares Means and Ratio Percents | (BB5800040) | (BB5800087) | (BB5800047) | Maximally | Skelaxin Reference |
| (Test/Skelaxin-Reference) of | Less Bioavailable | Bioequivalent | More Bioavailable Than | Bioavailable | Maximally Bioavailable |
| Cₘₐₓ, AUC_{inf} and AUC₀₋ₜ and Tₘₐₓ | Than Skelaxin | To Skelaxin | Skelaxin | Liquisolid Product | Liquisolid Product |
| Cₘₐₓ Means (Test & Ref): | (in ng/mL) | (in ng/mL) | (in ng/mL) | (in ng/mL) | (in ng/mL) |
| Cₘₐₓ of Test Product | 518 | 710 | 1,796 | 3,022 | 3,109 |
| Cₘₐₓ of Skelaxin Reference | 669 | 672 | 777 | 932 | 713 |
| (N = number of subjects-Fasting) | (N = 35) | (N = 43) | (N = 24) | (N = 8) | (Projected Values) |
| Cₘₐₓ Ratio % (Fasting) | 77.4% | 105.6% | 231.2% | 324.3% | 436.0% |
| (N = number of subjects-Fasting) | (N = 35) | (N = 43) | (N = 24) | (N = 8) | (Projected Value |
| {Lower-Upper 90% C.I. Limits} | {61.8%-93.0%} | {94.9%-116.4%} | {206%-257%} | {281%-368%} | based on Test-C |
| | | | | | dissolution data) |
| | (in ng-hr/mL) | (in ng-hr/mL) | (in ng-hr/mL) | (in ng-hr/mL) | (in ng-hr/mL) |
| AUC_{inf} Means (Test & Ref.): | | | | | |
| AUC_{inf} of Test Product | 4,569 | 4,990 | 8,223 | 11,130 | 12,664 |
| AUC_{inf} of Skelaxin Reference | 5,215 | 5,633 | 5,956 | 6,260 | 5,616 |
| (N = number of subjects-Fasting) | (N = 35) | (N = 43) | (N = 24) | (N = 8) | (Projected Values) |
| AUC_{inf} Ratio % (Fasting) | 87.6% | 88.6% | 138.1% | 177.8% | 225.5% |
| (N = number of subjects) | (N = 35) | (N = 43) | (N = 24) | (N = 8) | (Projected Value |
| {Lower-Upper 90% C.I. Limits} | {79.4%-95.8%} | {82.6%-94.6%} | {124%-154%} | {161%-195%} | based on Test-C |
| | | | | | dissolution data) |
| AUC₀₋ₜ Means (Test & Ref.): | | | | | |
| AUC₀₋ₜ of Test Product | 4,365 | 4,728 | 8,138 | 11,064 | 12,590 |
| AUC₀₋ₜ of Skelaxin Reference | 5,074 | 5,381 | 5,672 | 6,000 | 5,392 |
| (N = number of subjects-Fasting) | (N = 35) | (N = 43) | (N = 24) | (N = 8) | (Projected Values) |
| AUC₀₋ₜ Ratio % (Fasting) | 86.0% | 87.9% | 143.5% | 184.4% | 233.5% |
| (N = number of subjects) | (N = 35) | (N = 43) | (N = 24) | (N = 8) | (Projected Value |
| {Lower-Upper 90% C.I. Limits} | {77.9%-94.2%} | {81.7%-94.1%} | {129%-158%} | {167%-202%} | based on Test-C |
| | | | | | dissolution data) |
| Tₘₐₓ Means (Test & Ref.): | (in hours) | (in hours) | (in hours) | (in hours) | |
| Tₘₐₓ of Test Product | 3.69 | 3.50 | 3.02 | 2.94 | N/A |
| Tₘₐₓ of Skelaxin Reference | 3.43 | 4.07 | 3.40 | 3.94 | |
| (N = number of subjects-Fasting) | (N = 35) | (N = 43) | (N = 24) | (N = 8) | |
| Tₘₐₓ Ratio % (Fasting) | 107.6% | 86.1% | 88.8% | 74.5% | |
| (N = number of subjects) | (N = 35) | (N = 43) | (N = 24) | (N = 8) | N/A |
| {Lower-Upper 90% C.I. Limits} | {91.8%-124%} | {70.1%-102.8%} | {76%-102%} | {54%-95%} | |
| kₑₗᵢₘ Means (Test & Ref.): | (in hrs⁻¹) | (in hrs⁻¹) | (in hrs⁻¹) | (in hrs⁻¹) | |
| kₑₗᵢₘ of Test Product | 0.1116 | 0.1178 | 0.3794 | 0.4413 | N/A |
| kₑₗᵢₘ of Skelaxin Reference | 0.1157 | 0.1324 | 0.1081 | 0.1103 | |
| (N = number of subjects-Fasting) | (N = 35) | (N = 43) | (N = 24) | (N = 8) | |
| kₑₗᵢₘ Ratio % (Fasting) | 96.5% | 89.0% | 351.0% | 398.2% | |
| (N = number of subjects) | (N = 35) | (N = 43) | (N = 24) | (N = 8) | N/A |
| {Lower-Upper 90% C.I. Limits} | {84.6%-108%} | {74.3%-103.6%} | {296%-407%} | {338%-458%} | |
| t_{1/2} Means (Test & Ref.): | (in hours) | (in hours) | (in hours) | (in hours) | |
| t_{1/2} of Test Product | 7.75 | 7.58 | 2.46 | 1.64 | N/A |
| t_{1/2} of Skelaxin Reference | 6.66 | 6.13 | 7.66 | 6.75 | |
| (N = number of subjects-Fasting) | (N = 35) | (N = 43) | (N = 24) | (N = 8) | |
| t_{1/2} Ratio % (Fasting) | 116.4% | 123.5% | 32.1% | 24.3% | |
| (N = number of subjects) | (N = 35) | (N = 43) | (N = 24) | (N = 8) | N/A |
| {Lower-Upper 90% C.I. Limits} | {87.7%-145%} | {107%-140%} | {14%-51%} | {5%-44%} | |

TABLE 4
Comparison of oral bioavailability of metaxalone as defined by the geometric
means of the ln-transformed least squares means of critical pharmacokinetic
parameters and the ln-transformed geometric means of Test/Reference ratio
percents of Cₘₐₓ, AUC_{inf} and AUC₀₋ₜ obtained from clinical biostudies on healthy
human volunteers in two period crossover studies involving a single dose oral
administration under fasting and non-fasting conditions of each of the four
experimental metaxalone tablet formulations (Ex. #1-4) used as the Test product
versus Skelaxin ® 400-mg tablets used as the Reference product. The projected
values expected to be obtained from a similar bioequivalence study comparing the
fifth experimental tablet formulation (Ex. #5) against the Reference Skelaxin ®
400-mg tablets are also tabulated.
METAXALONE TABLET FORMULATIONS Tested Against SKELAXIN 400-mg Tablets

| Ln-Transformed Data: | | | | | |
|---|---|---|---|---|---|
| Geometric Means of | | | | | |
| ln-transformed Least | | | | Ex. # 4 | Ex. # 5 |
| Squares Means of Critical | | | | (BB5800056) | (NB1190:48) |
| PK Parameters and | | | | Significantly More | Projected to be |
| Test/Reference Ratios | | | | Bioavailable Than | Significantly More |
| Geometric Means of ln- | Ex. # 1 | Ex. # 2 | Ex. # 3 | Skelaxin Reference | Bioavailable Than |
| transformed Least Square Means | (BB5800040) | (BB5800087) | (BB5800047) | Maximally | Skelaxin Reference |
| and Ratio Percents (Test/Skelaxin) | Less Bioavailable | Bioequivalent | More Bioavailable Than | Bioavailable | Maximally Bioavailable |
| of Cₘₐₓ, AUC_{inf} and AUC₀₋ₜ | Than Skelaxin | To Skelaxin | Skelaxin | Liquisolid Product | Liquisolid Product |
| Cₘₐₓ Geometric Means: | (in ng/mL) | (in ng/mL) | (in ng/mL) | (in ng/mL) | (in ng/mL) |
| Cₘₐₓ of Test Product | 425 | 647 | 1,669 | 2,936 | 3,141 |
| Cₘₐₓ of Skelaxin Reference | 620 | 606 | 721 | 851 | 653 |
| (N = number of subjects-Fasting) | (N = 35) | (N = 43) | (N = 24) | (N = 8) | (Projected Values) |
| Cₘₐₓ Ratio % (Fasting) | 68.5% | 106.9% | 231.6% | 344.9% | 480.9% |
| (N = number of subjects) | (N = 35) | (N = 43) | (N = 24) | (N = 8) | (Projected Value |
| {Lower-Upper 90% C.I. Limits} | {55.5%-84.5%} | {95.8%-119.3%} | {202%-266%} | {263%-452%} | based on Test-C |
| | | | | | dissolution data) |
| | (in ng-hr/mL) | (in ng-hr/mL) | (in ng-hr/mL) | (in ng-hr/mL) | (in ng-hr/mL) |
| AUC_{inf} Geometric Means: | | | | | |
| AUC_{inf} of Test Product | 4,196 | 4,518 | 7,518 | 10,942 | 12,508 |
| AUC_{inf} of Skelaxin Reference | 4,939 | 5,107 | 5,453 | 5,920 | 5,188 |
| (N = number of subjects-Fasting) | (N = 35) | (N = 43) | (N = 24) | (N = 8) | (Projected Values) |
| AUC_{inf} Ratio % (Fasting) | 85.0% | 88.5% | 137.9% | 184.8% | 241.1% |
| (N = number of subjects) | (N = 35) | (N = 43) | (N = 24) | (N = 8) | (Projected Value |
| {Lower-Upper 90% C.I. Limits} | {77.2%-93.4%} | {83.5%-93.8%} | {124%-154%} | {157%-218%} | based on Test-C |
| | | | | | dissolution data) |
| AUC₀₋ₜ Geometric Means: | | | | | |
| AUC₀₋ₜ of Test Product | 3,932 | 4,267 | 7,428 | 10,872 | 12,400 |
| AUC₀₋ₜ of Skelaxin Reference | 4,784 | 4,875 | 5,162 | 5,661 | 4,966 |
| (N = number of subjects-Fasting) | (N = 35) | (N = 43) | (N = 24) | (N = 8) | (Projected Values) |
| AUC₀₋ₜ Ratio % (Fasting) | 85.0% | 88.5% | 143.9% | 192.0% | 249.7% |
| (N = number of subjects) | (N = 35) | (N = 43) | (N = 24) | (N = 8) | (Projected Value |
| {Lower-Upper 90% C.I. Limits} | {77.2%-93.4%} | {83.5%-93.8%} | {129%-161%} | {163%-226%} | based on Test-C |
| | | | | | dissolution data) |
| Cₘₐₓ Ratio % (Non-Fasting) | | 93.0% | | | |
| (N = number of subjects) | N/A | (N = 24) | N/A | N/A | N/A |
| {Lower-Upper 90% C.I. Limits} | | {74.7%-115.8%} | | | |
| AUC_{inf} Ratio % (Non-Fasting) | | 86.9% | | | |
| (N = number of subjects) | N/A | (N = 24) | N/A | N/A | N/A |
| {Lower-Upper 90% C.I. Limits} | | (80.0%-94.4%) | | | |
| AUC₀₋ₜ Ratio % (Non-Fasting) | | 90.1% | | | |
| (N = number of subjects) | N/A | (N = 24) | N/A | N/A | N/A |
| {Lower-Upper 90% C.I. Limits} | | (82.1%-98.9%) | | | |

Discussion of the Results:

As shown in Tables 2-4 above, Ex.#1 metaxalone tablet formulation (BB5800040) which demonstrated in all three dissolution tests slower drug dissolution rates than the Skelaxin® 400-mg tablets, was also found to be “less bioavailable” than the reference Skelaxin® product of NDA #13-217. Surprisingly, however, as shown in FIGS. 1-4 and Tables 2-4, even though Ex.#2 metaxalone tablet formulation (BB5800087) exhibited significantly faster drug dissolution rates than the reference Skelaxin® 400-mg tablets in Dissolution Test-A and Test-B, it was found to be “bioequivalent” to the Skelaxin® tablets of NDA #13-217. It is therefore possible for metaxalone tablets to be prepared which can display drug dissolution rates higher than those of their commercial Skelaxin® counterparts while at the same time demonstrating at both fasting and non-fasting conditions bioavailability properties similar to those of the reference Skelaxin® product of NDA #13-217. On the other hand, as shown in Tables 2-4, Ex.#3 metaxalone tablet formulation (BB5800047) which does not contain a nonvolatile liquid, demonstrated faster drug dissolution rates in all three dissolution tests than the reference Skelaxin® 400-mg tablets and was found to be “more bioavailable” than the reference Skelaxin® product of NDA #13-217.

Most importantly, however, Ex.#4 metaxalone liquisolid tablet formulation (BB5800056) which contains a nonvolatile liquid (i.e., polyethylene glycol 400), not only demonstrated faster drug dissolution rates in all three dissolution tests than Ex.#3 metaxalone tablets and the reference Skelaxin® 400-mg tablets but it was also found to be significantly-more-bioavailable than the reference Skelaxin® product of NDA #13-217. Furthermore, Ex.#5 metaxalone liquisolid tablets (NB1190:48) which contain larger amounts of another nonvolatile liquid (i.e., polysorbate 80) demonstrated faster drug dissolution rates than even the maximally bioavailable Ex.#4 metaxalone liquisolid tablet formulation. Mathematical projections and modeling based on the drug dissolution rates obtained from dissolution Test-C lead to the conclusion that Ex.#5 metaxalone should be expected to display Test/Reference Cₘₐₓ and AUC_{inf} ratio percents which would be even greater than those obtained from the maximally bioavailable Ex.#4 metaxalone tablets. Such projected maximally enhanced bioavailability properties of Ex.#5 as compared to those of the reference Skelaxin® product of NDA #13-217 are included in Tables 3 and 4.

In summary, based on the results presented herein, it is apparent that pharmaceutically acceptable solid dosage forms of an essentially water-insoluble drug such as metaxalone can be prepared using the ingredient compositions and manufacturing methods discussed herein. Such compositions demonstrate improved drug dissolution rates as compared to commercial lots of the essentially water-insoluble drug such as Skelaxin® 400-mg tablets, while at the same time being bioequivalent to, more bioavailable than, or significantly more bioavailable than conventionally made and/or commercially available reference products such as the Skelaxin® tablets of NDA #13-217.

As shown herein, metaxalone products significantly more bioavailable than the reference Skelaxin® tablets of NDA #13-217 can be prepared to contain various amounts of nonvolatile liquids using Liquisolid technology. Based on the methods, compositions, results and projections presented herein, it is apparent that such metaxalone Liquisolid tablets are maximally bioavailable thereby leading to new metaxalone dosage forms which, even if they contain smaller doses of metaxalone per unit dose as compared to the reference Skelaxin® 400-mg tablets of NDA #13-217 (say liquisolid metaxalone 50 mg to 350 mg tablets), they would still be bioequivalent to said Skelaxin® 400-mg tablets of NDA #13-217. Such maximally bioavailable low dose liquisolid metaxalone products represent unique, novel and commercially desirable products and are incorporated herein as one of the preferred embodiments of the present invention. Finally, it should be also expected that metaxalone products prepared according to the present invention and demonstrating maximally enhanced bioavailability properties under fasting conditions as compared to the Skelaxin® tablets, would not present any food effects on oral bioavailability, i.e., they should demonstrate similar oral bioavailability under both fasting and non-fasting dosing conditions.

The foregoing discussion presented several examples of specific embodiments for carrying out the present invention. The examples are offered for illustrative purposes only, and are not intended to limit the scope of the present invention in any way.

All publications, patents and patent applications cited herein are hereby incorporated by reference in their entirety. As used in this specification and the appended claims, the singular forms “a,” “an” and “the” include plural references unless the content clearly dictates otherwise. Thus, for example, reference to “an excipient” includes a mixture of two or more excipients.

Other aspects of the invention will be apparent from review of the present specification and claims and all such falling within the spirit of the invention are comprehended hereby.

1-36. (canceled)

37. A pharmaceutical solid dosage form comprising an effective amount of metaxalone and at least one inactive powder excipient, wherein:
(a) said dosage form contains at least one nonvolatile liquid; and
(b) said dosage form presents improved drug dissolution rate as compared to the metaxalone product of New Drug Application No. 13-217 (“NDA #13-217”).

38. The pharmaceutical solid dosage form of claim 37, wherein said dosage form is bioequivalent to the metaxalone product of NDA #13-217 upon oral administration to a fasting or non-fasting subject.

39. The pharmaceutical solid dosage form of claim 37, wherein said dosage form is more bioavailable than the metaxalone product of NDA #13-217 upon oral administration to a fasting or non-fasting subject.

40. A pharmaceutical solid dosage form comprising metaxalone and at least one inactive powder excipient, wherein:
(a) said dosage form contains at least one nonvolatile liquid; and
(b) further wherein:
(i) 13% by weight or greater of said metaxalone is dissolved about 30 minutes after said dosage form is placed in a peak glass dissolution vessel filled with 1000 mL of purified water maintained at 25° C. and stirred at a paddle speed of 100 rpm using a USP Type II (paddle) apparatus; or
(ii) 28% by weight or greater of said metaxalone is dissolved about 30 minutes after said dosage form is placed in a peak glass dissolution vessel filled with 1000 mL of purified water maintained at 35° C. and stirred at a paddle speed of 100 rpm using a USP Type II (paddle) apparatus; or
(iii) 27% by weight or greater of said metaxalone is dissolved about 30 minutes after said dosage form is placed in a peak glass dissolution vessel filled with 500 mL of an aqueous solution of 0.1% by weight of Sodium Lauryl Sulfate per volume of water, maintained at 37° C. and stirred at a paddle speed of 50 rpm using a USP Type II (paddle) apparatus.

41. The pharmaceutical solid dosage form of any one of claims 37 to 40 in which said dosage form comprises at least one inactive powder excipient selected from the group consisting of sodium alginate, ammonium alginate, calcium alginate, sodium carboxymethyl cellulose, hydroxypropyl cellulose, hydroxypropylmethyl cellulose, ethyl cellulose, microcrystalline cellulose, powder cellulose, amorphous cellulose, pregelatinized starch, corn starch, maize starch, potato starch, sodium starch glycolate, lactose, sucrose, maltose, dextrose, agarose, cyclodextrins, polyvinyl pyrrolidones, methacrylic acid, methacrylic acid copolymers, dicalcium phosphate, tricalcium phosphate, amorphous silicon dioxide, talc, waxes, and combinations thereof.

42. A method of making a pharmaceutical solid dosage form of any one of claims 37 to 40 comprising compounding metaxalone with one or more inactive powder excipients, wherein said dosage form contains at least one nonvolatile liquid.

43. A method of making a pharmaceutical solid dosage form of claim 41 comprising compounding metaxalone with one or more inactive powder excipients, wherein said dosage form contains at least one nonvolatile liquid.
